(19) United States
(12) Patent Application Publication
(10) Pub. No.: US 2009/0270821 A1
(43) Pub. Date: Oct. 29, 2009

(54) ANGLED OPHTHALMIC DROPPER TIP

(75) Inventors: Don F. Yeager, Millville, NJ (US); Paul H. Andrews, Millville, NJ (US)

Correspondence Address: RATNERPRESTIA, P.O. BOX 980, VALLEY FORGE, PA, 19482, US

(73) Assignee: Alcan Global Pharmaceutical Packaging , Inc., Pennsauken, NJ (US)

(21) Appl. No.: 12/306,409

(22) PCT Filed: Jun. 27, 2007
(86) PCT No.: PCT/US2007/014926

§ 371 Date: May 13, 2009

Related U.S. Application Data
(60) Provisional application No. 60/817,129, filed on Jun. 28, 2006.

(51) Int. Cl. A61F 9/00

(52) U.S. Cl. 604/298

(57) ABSTRACT

An ophthalmic dropper for distributing medicinal fluid to an eye is provided. The dropper comprises a fluid carrying container having a hollow body extending along a longitudinal axis, and an opening defined in the hollow body. A nozzle is coupled to the opening of the container for receiving fluid from the container. The nozzle defines an outlet port for distributing fluid from the nozzle. The outlet port of the nozzle is positioned to deliver fluid along an axis that is substantially perpendicular to the longitudinal axis of the container.

### FIELD OF THE INVENTION

The present invention relates to eye dropper bottles for dispensing fluids. More particularly, this invention relates to bottles for instilling medicinal fluids into a user's eye.

### BACKGROUND OF THE INVENTION

The instillation of medicinal eye drops tends to be difficult and perilous for many individuals. Generally, a user tilts their head back and looks up. Simultaneously, the dropper bottle is elevated above the eye and held in an inverted position while the walls of the bottle are squeezed, causing the drop to fall from the tip of the bottle toward the eye.

There are several factors that often complicate the conventional way of instilling eye drops. First, some individuals, as they grow older, find that their hands and head are no longer steady, thus posing the problem of not being able to maintain proper alignment while the drops are being instilled. The individual may even unintentionally poke themselves in the eye with the tip of the dropper, causing temporary or permanent damage to the eye. Secondly, it is difficult for some individuals, especially the elderly, to elevate their shoulder high enough to place the eye dropper in an ideal position above the eye. Thirdly, limitation of motion of the hand or the wrist makes it difficult to turn the bottle in a substantially inverted position.

Accordingly, there continues to be a need to further develop and improve eye dropper bottles in the interests of safety and convenience.

### SUMMARY OF THE INVENTION

According to one aspect of the invention, an ophthalmic dropper is provided. The ophthalmic dropper comprises a fluid carrying container having a hollow body extending along a longitudinal axis, and an opening defined in the hollow body. A nozzle is coupled to the opening of the container for receiving fluid from the container. The nozzle defines an outlet port for distributing fluid from the nozzle, wherein the outlet port of the nozzle is positioned to deliver fluid along an axis that is substantially perpendicular to the longitudinal axis of the container.

According to another aspect of the invention, the nozzle of the ophthalmic dropper includes a mounting portion for coupling to the opening of the container and receiving fluid from the container. A side surface of the nozzle extends above the mounting portion of the nozzle in a direction parallel to the opening of the container. The outlet port of the nozzle is disposed on the side surface for distributing fluid from the nozzle.

According to yet another aspect of the invention, the ophthalmic dropper further comprises a closure moveably captivated to the nozzle. The closure is moveable with respect to the nozzle between a retracted position and an extended position. In the retracted position at least a portion of the nozzle is positioned over the outlet port of the nozzle for concealing the outlet port. In the extended position the nozzle is spaced from the outlet port of the nozzle for distributing fluid through the outlet port.

### BRIEF DESCRIPTION OF THE DRAWINGS

The invention is best understood from the following detailed description when read in connection with the accompanying drawing. It is emphasized that, according to common practice, the various features of the drawing may not be to scale. On the contrary, the dimensions of the various features may be arbitrarily expanded or reduced for clarity. Included in the drawing is the following figure:

FIG. 1A is a cross-sectional elevation view of an ophthalmic dropper according to a first exemplary embodiment of the invention;

FIG. 1B is a top plan view of the nozzle shown in FIG. 1A, whereby the restrictor channel is shown in hidden lines;

FIG. 1C is a cross sectional view of the nozzle shown in FIG. 1B taken along the lines 1C-1C;

FIG. 1D is a side elevation view of the closure shown in FIG. 1A;

FIG. 1E is a top plan view of the closure shown in FIG. 1D;

FIG. 1F is a cross sectional view of the closure shown in FIG. 1E taken along the lines 1F-1F;

FIG. 2A is a cross-sectional elevation view of an ophthalmic dropper according to a second exemplary embodiment of the invention;

FIG. 2B is a top plan view of the nozzle shown in FIG. 2A, whereby the restrictor channel is shown in hidden lines;

FIG. 2C is a cross sectional view of the nozzle shown in FIG. 2B taken along the lines 2C-2C;

FIG. 2D is a top plan view of the closure shown in FIG. 2A;

FIG. 2E is a cross sectional view of the closure shown in FIG. 2D taken along the lines 2E-2E;

FIG. 3A is a cross-sectional elevation view of an ophthalmic dropper according to a third exemplary embodiment of the invention (container omitted), wherein the closure is oriented in a retracted position;

FIG. 3B is a cross-sectional elevation view of the ophthalmic dropper of FIG. 3A (container omitted), wherein the closure is oriented in an extended position;

FIG. 3C is a top plan view of the closure shown in FIG. 3A;

FIG. 3D is a cross sectional view of the closure shown in FIG. 3C taken along the lines 3D-3D;

FIG. 3E is a top plan view of the nozzle shown in FIG. 3A, whereby the restrictor channel is shown in hidden lines;

FIG. 3F is a cross sectional view of the nozzle shown in FIG. 3E taken along the lines 3F-3F;

FIG. 3G is a top plan view of another nozzle that is adapted to be mounted to the closure shown in FIG. 3A, whereby the restrictor channel is shown in hidden lines; and

FIG. 3H is a cross sectional view of the nozzle shown in FIG. 3G taken along the lines 3H-3H.

### DETAILED DESCRIPTION OF THE INVENTION

The invention is best understood from the following detailed description when read in connection with the accompanying drawing, which shows exemplary embodiments of the invention selected for illustrative purposes. The invention will be illustrated with reference to the Figures. Such Figures are intended to be illustrative rather than limiting and are included herewith to facilitate the explanation of the present invention. In the various embodiments like item numbers represent substantially similar features.

In conventional eye dropper containers, such as Visine® and Clear Eyes®, a fluid nozzle is positioned at a blunt end of the container. In practice, a user tilts the head back, inverts the conventional dropper 180 degrees relative to the upright position to induce the flow of fluid through the eye dropper, and presents the blunt end of the container straight toward the eye. As described in the background section, this method of hydrating and/or medicating the eye presents a risk of poking injury to the eye. The invention described herein represents a significant improvement over the conventional eye droppers in the interest of safety.

Referring generally to the exemplary droppers 10, 110, 210 illustrated in the drawing figures, dropper 10, 110, 210 generally includes container 15, 115 for storing fluid, a nozzle 20, 120, 220A, 220B coupled to the container for controllably releasing the fluid, and a closure 25, 125, 225 releasably fastened to the container or nozzle.

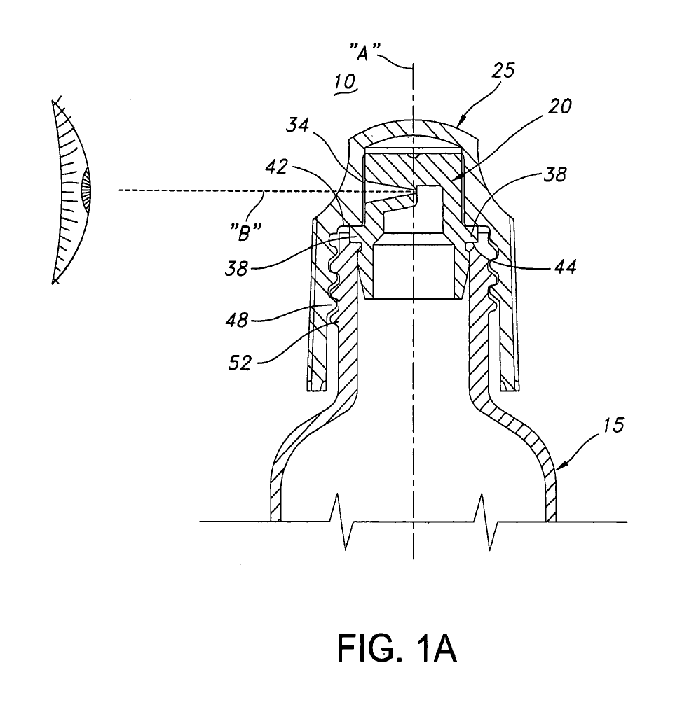

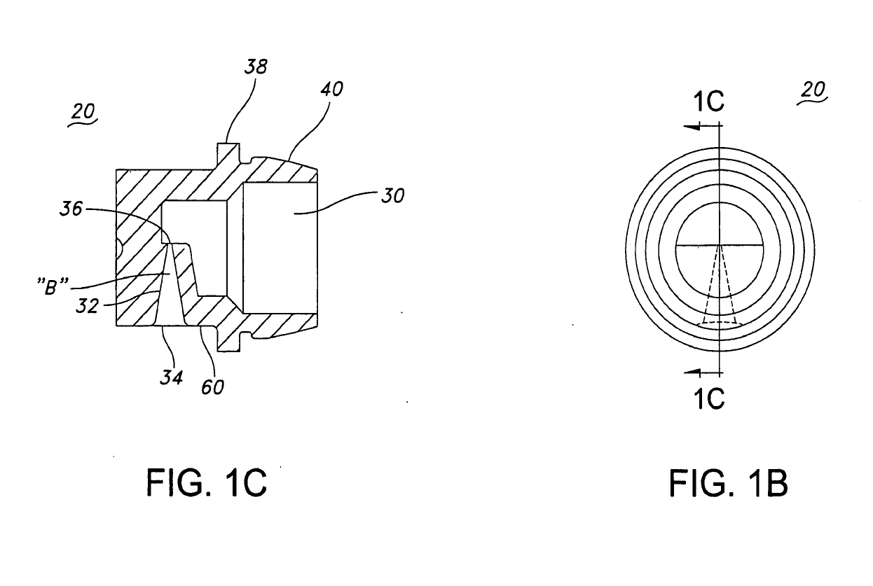

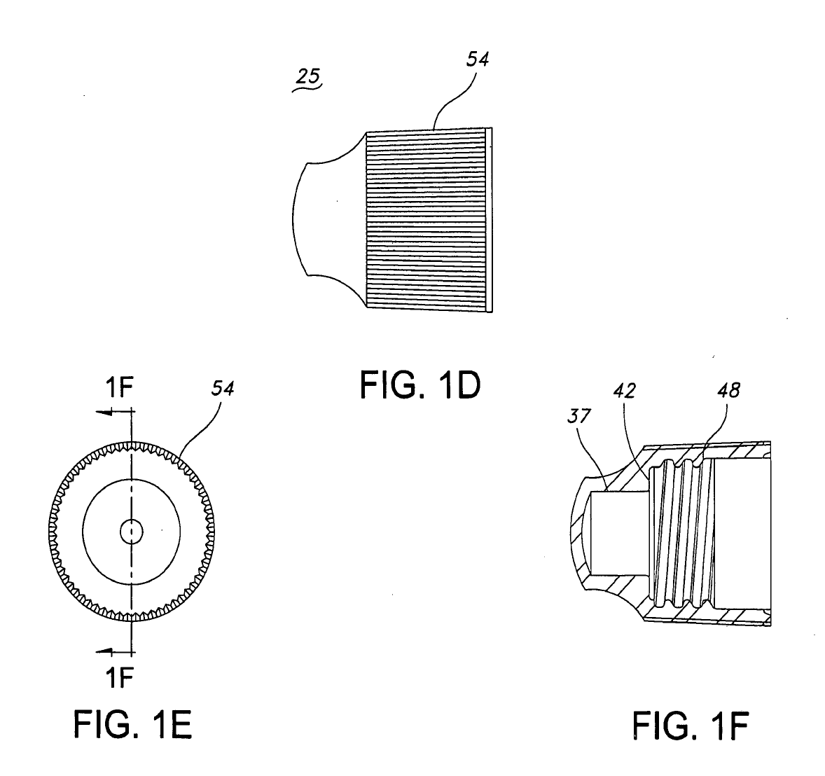

In practice, a user rotates the dropper 10, 110, 210 approximately 90 degrees relative to an upright position to orient the dropper across the front of the eye (eye droppers are generally stored in an upright position). In other words, the body of the dropper is oriented orthogonal to the nose and parallel to the width of the eye, as best illustrated in FIG. 1A. The orientation of the eye dropper with respect to the direction of the eye lashes shown in FIG. 1A should be noted. The fluid exits from an outlet port 34, 134, 234 forming droplets at a side surface 60, 160, 260A, 260B of the dropper as opposed to a top surface of the dropper. Positioning the outlet port on the side surface of the dropper permits a user to orient the dropper nozzle across the front of the eye. It has been discovered that presenting a dropper across the front of the eye, as opposed to presenting the dropper straight toward the eye, reduces the possibility of poking injury to the eye. It is believed that this aspect of the invention represents a significant improvement over conventional eye droppers.

Referring specifically to the exemplary embodiment illustrated in FIGS. 1A through 1F, a dropper according to one aspect of this invention is generally designated by the numeral “10.” The dropper 10 is shown in a substantially horizontal orientation relative to the eye. In other words, dropper 10 is rotated 90 degrees with respect to an upright position. The dropper includes container 15, a nozzle 20 coupled to container 15, and a closure 25 threadedly fastened to container 15. In FIG. 1A, closure 25 is shown fastened to the top end, otherwise known as a finish, of container 15. In the exemplary embodiment shown, container 15 has a threaded finish. A portion of container 15 is illustrated in FIG. 1A. Detailed views of nozzle 20 are illustrated in FIGS. 1B and 1C, and detailed views of closure 25 are illustrated in FIGS. 1D through 1F.

The container 15 holds a pre-determined volume of fluid, such as medicine, saline solution, water, air or any other fluid adapted for use with an eye. The container 15 defines a longitudinal axis “A,” but is not limited to any particular size or shape. The container 15 may be formed from a flexible material, such as polyethylene, for example, such that compression of container 15 by a user induces the fluid within container 15 to flow towards nozzle 20. Alternatively, container 15 may be formed from a substantially rigid material, such as glass.

When dropper 10 is rotated away from the upright position or compressed, the fluid within container 15 flows toward nozzle 20. The nozzle 20 is adapted to controllably release the fluid from dropper 10 (assuming closure 25 is removed from container 15). More specifically, as best shown in FIG. 1C, fluid from dropper 10 is introduced through an annular passageway 30 formed in nozzle 20. Thereafter the fluid gradually travels through a restrictor channel 32 formed in the body of nozzle 20. The restrictor channel 32 spans from annular passageway 30 to an exterior side surface 60 of nozzle 20. The restrictor channel 32 is adapted to permit a controlled volume of fluid to pass through nozzle 20. The fluid travels through a small aperture 36 formed at one end of the conically shaped fluid restrictor channel 32 and exits nozzle 20 through the opposing end of restrictor channel 32 at outlet port 34 that is formed on side surface 60 of nozzle 20. The diameters of aperture 36 and outlet port 34 are precisely tailored to control the volume and flow rate of the fluid through nozzle 20.

The fluid is ultimately delivered through outlet port 34 along axis “B”. According to this exemplary embodiment, axis “B” is substantially orthogonal to the longitudinal axis “A” of the container 15, as best illustrated in FIG. 1A. However, axis “B” may be disposed at any other angle with respect to the longitudinal axis “A” of the container 15.

Referring still to FIGS. 1A through 1F, when closure 25 is fastened to container 15, as shown in FIG. 1A, closure 25 conceals outlet port 34 of nozzle 20 to prevent or limit the escapement of fluid from dropper 10. More specifically, once closure 25 is seated with flange 38 of nozzle 20, the interior surface 37 of closure 25 (see FIG. 1F) conceals the outlet port 34 of nozzle 20. Thus, if dropper 10 is unintentionally stored on its side, physical contact between interior surface 37 and outlet port 34 limits escapement of fluid from outlet port 34. In addition, because both the interior surface 37 of closure 25 and nozzle 20 are cylindrical features, surface 37 conceals outlet port 34 regardless of the radial orientation of outlet port 34 or closure 25.

In assembly of dropper 10, a plug-shaped mounting portion 40 of nozzle 20 (see FIG. 1C) is inserted through an opening of container 15. Similar to a common plug, mounting portion 40 includes a tapered exterior that progressively engages the opening of container 15 until flange 38 of nozzle 20 bears on shoulder 44 of container 15. The outer revolved surface of mounting portion 40 is sufficiently compressed in the aperture of container 15 to limit escapement of fluid through the interface of the threaded finish of container 15 and flange 38 of the nozzle 20.

After nozzle 20 is coupled to container 15, closure 25 is positioned over nozzle 20 and threadedly fastened to container 15. Specifically, threaded region 48 of closure 25 (see FIG. 1F) is threadedly engaged with threaded region 52 of container 15 and the shoulder 42 of closure 25 is seated on flange 38 of nozzle 20. The exterior revolved surface of closure 25 includes ergonomic features, such as serrations 54, which facilitate easy fastening and release of closure 25 onto container 15. In this exemplary embodiment, the closure 25 may be entirely removed from dropper 10.

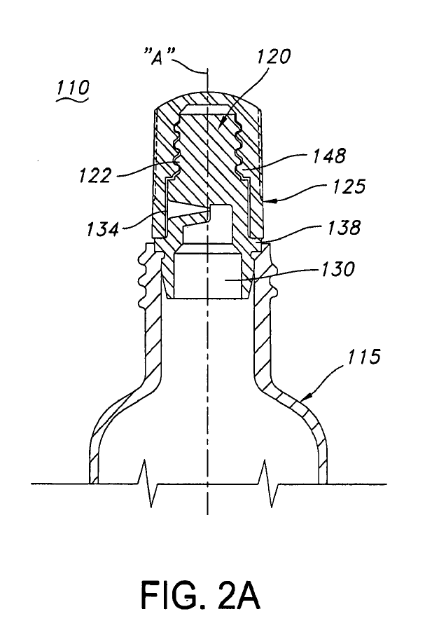

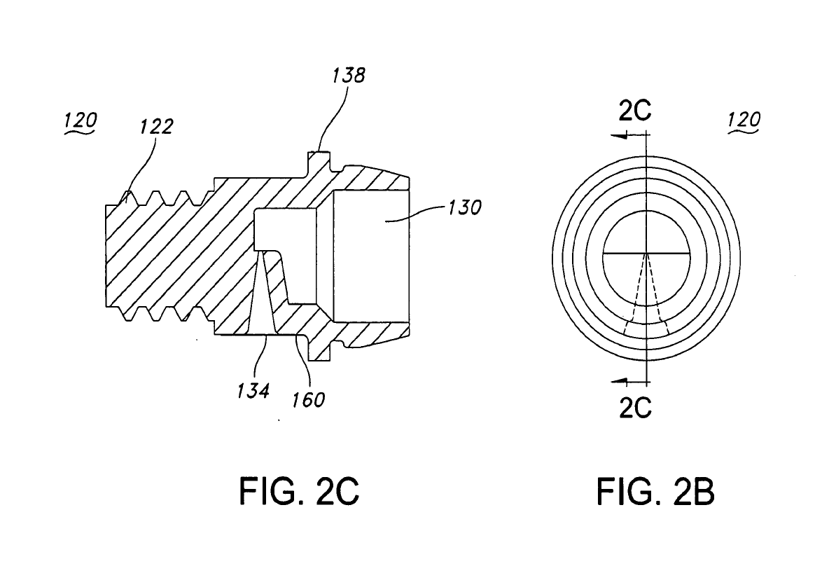

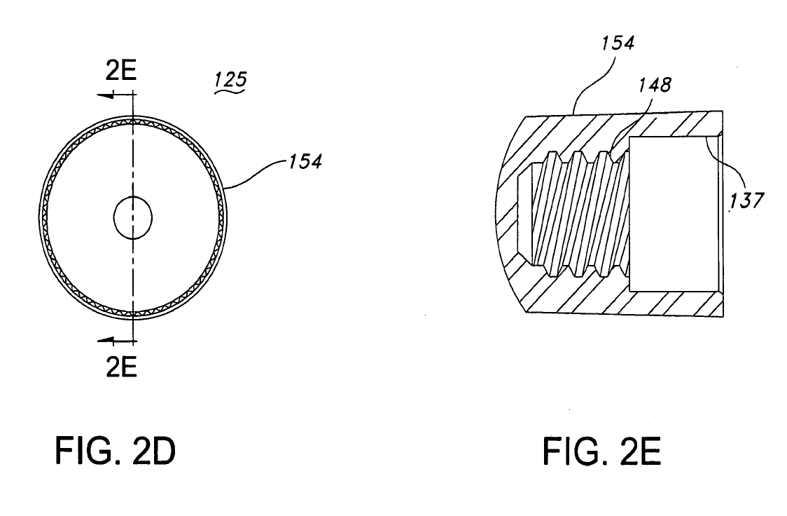

Referring now to FIGS. 2A through 2E, another exemplary embodiment of a dropper 110 is illustrated. The dropper 110 is illustrated in a closed configuration in FIG. 2A, i.e., outlet port 134 is concealed. Similar to the previous embodiment, the fluid exits from an outlet port 134 disposed on a side surface 160 (see FIG. 2C) of dropper 110, such that, in practice, dropper 110 is presented across the front of the eye. However, in this embodiment, closure 125 is threadedly fastened to nozzle 120, as opposed to container 115.

In this exemplary embodiment, dropper 110 comprises container 115, a nozzle 120 coupled to container 115, and a closure 125 threadedly fastened to nozzle 120. Detailed views of nozzle 120 are illustrated in FIGS. 2B and 2C, and detailed views of closure 25 are illustrated in FIGS. 2D and 2E.

In the retracted position of closure 125, the threaded region 148 of closure 125 is threadedly engaged with threaded region 122 of nozzle 120 and closure 125 bears on flange 138 of nozzle 120. The interior surface 137 of closure 125 (see FIG. 2E) conceals and is compressed against outlet port 134 of nozzle 120 to limit escapement of fluid from dropper 110. The exterior revolved surface of closure 125 includes serrations 154 to facilitate easy fastening and release of closure 125 onto dropper 110.

The closure 125 is configured to travel along axis “A”, from a retracted position, as shown in FIG. 2A, to an extended position (not shown). Specifically, closure 225 translates in an axial direction along axis “A” as it is rotated in either a clockwise or counterclockwise direction about axis “A.” In this exemplary embodiment, closure 125 may be entirely removed from dropper 110.

According to one exemplary use of the invention, the closure 125 is rotated to an extended position and is placed on the bridge of a user's nose. The outlet port 134 is then positioned directly adjacent the user's eye. The container 115 is squeezed to deliver fluid through the outlet port 134 of the nozzle 120 and into the user's eye.

By contacting the nose before positioning outlet port 134 directly adjacent the eye, a user can position outlet port 234 adjacent the eye with greater control and precision as compared with conventional droppers, thereby reducing the possibility of poking injury to the eye. Thus, it is submitted that this invention confers a significant advantage over conventional eye droppers.

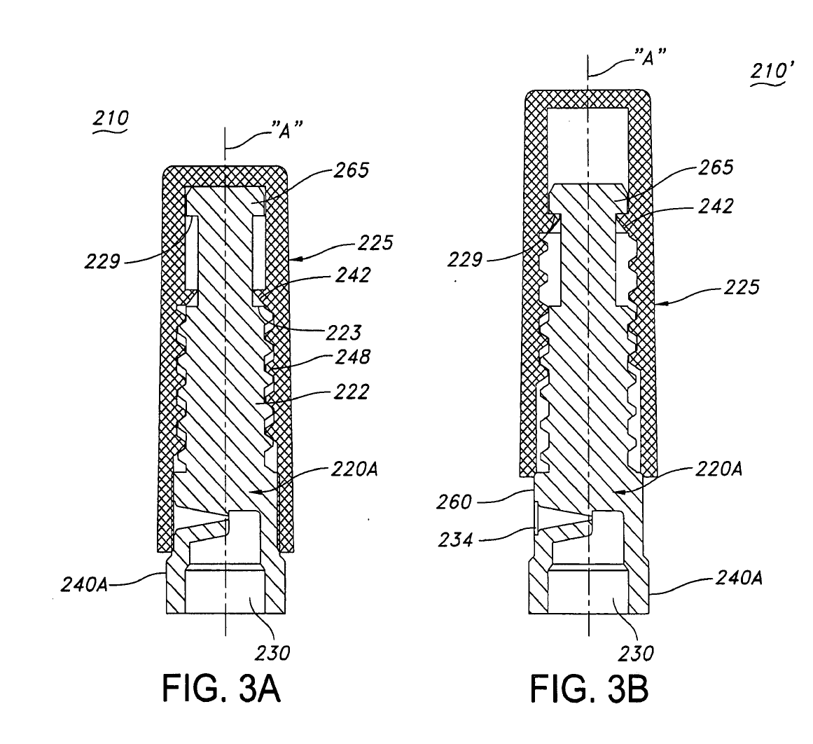

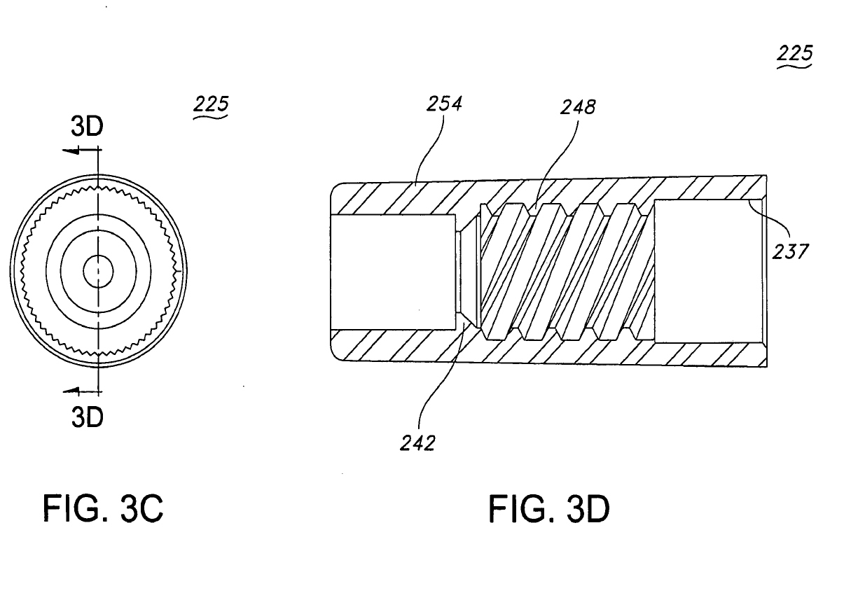

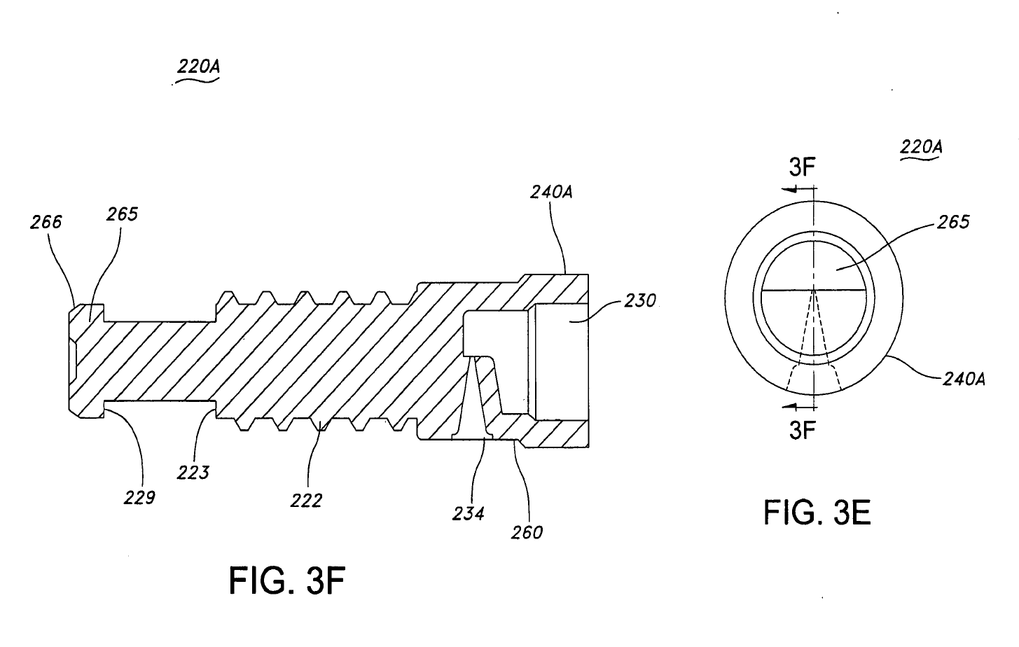

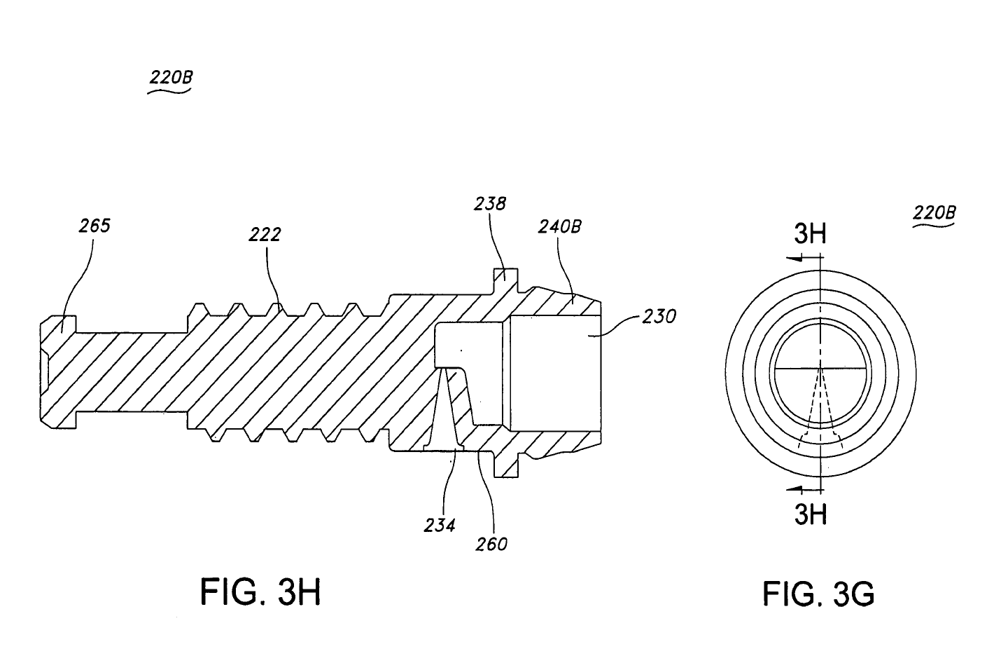

Referring now to FIGS. 3A through 3F, another exemplary embodiment of a dropper 210, 210′ (container omitted) is illustrated according to aspects of the invention. In FIG. 3A dropper 210 is illustrated in a closed configuration, and in FIG. 3B dropper 210′ is illustrated in an open configuration. The container of dropper 210 is omitted from FIGS. 3A and 3B.

The dropper 210, 210′ includes a container (not shown), a nozzle 220A coupled to the container, and a closure 225 threadedly fastened to nozzle 220A. In this exemplary embodiment, closure 225 is moveably captivated to nozzle 220A, i.e., closure 225 can not be removed from nozzle 220A without applying significant force.

The closure 225 is configured to travel along axis “A” between a retracted position and an extended position. The closure 225 of dropper 210 is illustrated in a retracted position in FIG. 3A, and the closure 225 of dropper 210′ is illustrated in an extended position in FIG. 3B. Similar to prior embodiments, in the extended position shown in FIG. 3B, closure 225 is spaced from outlet port 234 to permit escapement of fluid from the container. The fluid exits from an outlet port 234 disposed on a side surface 260 of the dropper 210′. Thus, it should be understood that dropper 210′ is utilized to medicate or hydrate a user's eye in the extended, i.e. open position, shown in FIG. 3B. In the retracted, i.e. closed, position shown in FIG. 3A, closure 225 conceals outlet port 234 of nozzle 220A to limit escapement of fluid from dropper 210.

The closure 225 is adapted to translate in an axial direction along axis “A” as it is rotated in either a clockwise or counterclockwise direction about axis “A.” Specifically, the threaded region 248 of closure 225 is threadedly engaged with threaded region 222 of nozzle 220A. The angled flange 242 of closure 225 is positioned to travel between shoulder 223 and shoulder 229 of nozzle 220A (see FIG. 3F).

To close dropper 210 and conceal outlet port 234, closure 225 is rotated about axis “A” until angled flange 242 of closure 225 is seated on shoulder 223 of nozzle 220A, as shown in FIG. 3A. Similar to prior embodiments, once closure 225 is seated on nozzle 220, interior surface 237 of closure 225 (see FIG. 3D) conceals the outlet port 234 of nozzle 220 to limit escapement of fluid from the container. Moreover, interior surface 237 of closure 225 is positioned in compressive contact with the outlet port 234, regardless of the radial orientation of outlet port 234 or closure 225.

Moreover, to open dropper 210 and expose outlet port 234, closure 225 is rotated about axis “A” in an opposite direction until angled flange 242 of closure 225 bears on shoulder 229 of nozzle 220A, as shown in FIG. 3B. However, it should be understood that dropper 210 is generally positioned in an open configuration (and permits escapement of fluid from the container) once closure 225 is spaced from outlet port 234 sufficient to expose outlet port 234. In other words, angled flange 242 of closure 225 does not have to bear on shoulder 229 of nozzle 220A to expose outlet port 234.

In this exemplary embodiment, the body of closure 225 extends substantially above the outlet 234. In practice, in the extended orientation of closure 225 shown in FIG. 3B, closure 225 is positioned on the bridge of a user's nose, and outlet port 234 is positioned directly adjacent the user's eye.

In assembly of dropper 210, 210′, mounting portion 240A of nozzle 220 is adhered to the container finish (not shown). Specifically, mounting portion 240A of nozzle 220 (see FIG. 3F) is positioned through an aperture defined in the container and either heat sealed, glued, or otherwise bonded to the cylindrical interior wall of the aperture. Thereafter, stop 265 of nozzle 220 is positioned through interior surface 237 of closure 225 (see FIG. 3D) and threadedly engaged with threaded region 248 of closure 225. As closure 225 is threaded onto nozzle 220A, chamfered surface 266 of stop 265 (see FIG. 3F) engages the angled surface of angled flange 242 to facilitate outward deflection of angled flange 242. Ultimately, stop 265 deflects angled flange 242 sufficient to permit the passage of stop 265 through angled flange 242, as shown in FIG. 3B. Thereafter, closure 225 is captivated onto dropper 210. To facilitate deflection of angled flange 242, either all or a portion of either closure 225 or nozzle 220 may be formed from a flexible or semi-flexible material. The method of assembling dropper 210 is not limited to the aforementioned steps, as the components of dropper 210 may be assembled in any sequence.

Referring now to FIGS. 3G and 3H, another example of a nozzle 220B adapted for use with dropper 210, 210′ is shown. The nozzle 220B shown in the figures includes a plug-shaped mounting portion 240B, similar to mounting portion 40 shown in FIG. 1C. In assembly, mounting portion 240B is inserted through an opening of the container (not shown). The mounting portion 240B includes a tapered exterior that progressively engages the opening of the container until flange 238 of nozzle 220B is seated on the shoulder of the container.

While preferred embodiments of the invention have been described herein, it will be understood that such embodiments are provided by way of example only. Numerous variations, changes and substitutions will occur to those skilled in the art without departing from the spirit of the invention. For example, although the closure is threadedly coupled to the nozzle or the container, the closure may be mounted to the nozzle or container using any fastening method known in the art.

It is intended that the appended claims cover all such variations as fall within the spirit and scope of the invention.

1.-20. (canceled)

21. An ophthalmic dropper comprising:
a fluid carrying container having a hollow body extending along a longitudinal axis, and an opening defined in the hollow body;
a nozzle coupled to the opening of the container for receiving fluid from the container, the nozzle including an outlet port positioned to deliver fluid from the container along an axis (B) that is substantially perpendicular to the longitudinal axis (A) of the container, said outlet port comprising a fluid passageway in fluid communication with the opening of the container,
wherein said outlet port comprises a fluid restrictor channel spanning from said passageway to an exterior side surface of said nozzle and having a shape adapted to permit a controlled volume of fluid to pass through the nozzle.

22. An ophthalmic dropper according to claim 21, wherein said fluid restrictor channel is conically shaped, with a small aperture defined at one end of the fluid restrictor channel and said outlet port being defined at an opposing end of the fluid restrictor channel and formed on the exterior side surface of the nozzle.

23. An ophthalmic dropper according to claim 22, wherein the diameters of said small aperture and said outlet port are tailored to control the volume and flow rate of fluid through the nozzle.

24. An ophthalmic dropper according to claim 23, further comprising a flange extending from the nozzle, wherein the flange is positionable against the opening of the container for limiting escapement of fluid at an interface between the nozzle and the container.

25. An ophthalmic dropper according to claim 24, wherein the nozzle has a mounting portion for coupling to the opening of the container and receiving fluid from the container.

26. An ophthalmic dropper according to claim 25, further comprising a side surface extending from the mounting portion of the nozzle in a direction parallel to the longitudinal axis (A) of the container, and an outlet port disposed on the side surface for distributing fluid from the nozzle.

27. An ophthalmic dropper according to claim 21, wherein the hollow body of the container is compressible for delivering fluid from the container through the outlet port of the nozzle.

28. An ophthalmic dropper according to claim 21, further comprising a movable closure for selectively exposing the outlet port of the nozzle.

29. An ophthalmic dropper according to claim 28, wherein the movable closure is coupled to the fluid carrying container.

30. An ophthalmic dropper according to claim 28, wherein the movable closure is coupled to the nozzle.

31. An ophthalmic dropper according to claim 30, wherein the moveable closure is moveably captivated to the nozzle.

32. An ophthalmic dropper according to claim 31, wherein the closure is moveable with respect to the outlet port of the nozzle between a retracted position and an extended position, wherein, in the retracted position, at least a portion of the closure is positioned over the outlet port of the nozzle for concealing the outlet port, and, in the extended position, the closure is spaced from the outlet port of the nozzle for distributing fluid through the outlet port.

33. An ophthalmic dropper according to claim 28, wherein the closure is moveable along the longitudinal axis (A) of the container.

34. An ophthalmic dropper according to claim 30, wherein the nozzle further comprises an engagement region for coupling with the closure.

35. An ophthalmic dropper according to claim 34, wherein the engagement region of the nozzle includes screw threads for coupling with screw threads disposed on the closure.
